(19) United States
(12) Patent Application Publication
(10) Pub. No.: US 2018/0153145 A1
(43) Pub. Date: Jun. 7, 2018

(54) HUMANIZED MICE AND USES THEREOF

(71) Applicant: Champions Oncology, Inc, HACKENSACK, NJ (US)

(72) Inventors: Baia S GILSON, San Francisco, CA (US); Keren PAZ, Tenafly, NJ (US); David SIDRANSKY, Pikesville, MD (US)

(73) Assignee: Champions Oncology, Inc., HACKENSACK, NJ (US)

(21) Appl. No.: 15/576,349

(22) PCT Filed: May 20, 2016
(86) PCT No.: PCT/US16/33562

§ 371 Date: Nov. 22, 2017

Related U.S. Application Data
(60) Provisional application No. 62/165,464, filed on May 22, 2015.

(51) Int. Cl. A01K 67/027; A61K 49/00

(52) U.S. Cl. CPC A01K 67/0271; A01K 2267/0331; A01K 2207/12; A61K 49/0008

(57) ABSTRACT

The invention relates to methods for generating, expanding and maintaining a culture of leukocytes in heterologous animals. The invention also relates to the use of these animals as models of human immune system for testing molecules in order to treat a disease or disorder such as cancer.

### CROSS-REFERENCE TO RELATED APPLICATIONS

This application claims priority to and the benefit of U.S. Provisional Patent Application 62/165,464, filed May 22, 2015, which is incorporated by reference herein in its entirety.

### FIELD OF THE INVENTION

The invention relates to methods for generating, expanding and maintaining a culture of leukocytes in heterologous animals. The invention also relates to the use of these animals as models of human immune system for testing molecules in order to treat a disease or disorder.

### BACKGROUND OF THE INVENTION

The spleen is the largest secondary lymphoid organ containing about one-fourth of the body's lymphocytes. The splenic subsets comprise of cells of the myeloid lineage, including dendritic cells and macrophages. In addition, in rodents extra medullary hematopoiesis is also present in the spleens and a minor fraction (<1%) of human CD34+ progenitor cells can be identified in splenocyte preps of humanized mice.

Adoptive cell therapy is a therapeutic approach comprising administration of a patient's own (autologous) or donor (allogeneic) anti-tumor or anti-pathogen lymphocytes, following a lymphodepleting preparative regimen. This approach has emerged as a potentially powerful tool of controlling pathological conditions, including infections and cancers. It also allows for generation of populations of lymphocytes with desired anti-pathogen specificity, which then can be available for use in case of recurrence of the pathology. The early protocols of adoptive transfer therapy selected the cells of desired specificity (e.g. anti-tumor leukocytes) and expanded them in the tissue culture. This approach, however, has significant limitations, including clonal selection in tissue culture, requirement for expensive tissue culture maintenance facilities, and limited scale-up potential. These concerns were partially addressed through the development of in vivo adoptive transfer protocol, which used immunodeficient animals, such as mice, to generate and maintain cultures of lymphocytes specific for a pathogen of choice. In case of cancer one protocol typically involves implantation of tumors into immunodeficient recipient animal (e.g. mouse) that has been “humanized” with xenograft of human cord blood-derived CD34+ hematopoietic stem cells (HSCs). This method however is limited by the availability of CD34+ HSCs. Furthermore the presence of tumor tissue in the humanized mouse limits scale-up potential and gives rise to safety concerns, since the resulting anti-tumor leukocyte population may also contain tumor cells. Another protocol involves implantation of tumor tissue into immunodeficient mice followed by expansion and subsequent harvesting of leukocytes that were co-implanted with tumor. While this method addresses the issue of limited availability of human cord blood-derived CD34+ HSCs, it does not resolve the limited scalability and safety concerns.

Accordingly, there exists a need for an improved adoptive transfer therapy.

### SUMMARY OF THE INVENTION

The present invention meets the aforementioned need by providing a method of maintaining and expanding a culture of human leukocytes in vivo.

In one aspect, the invention relates to a method for establishing a human immune system in a non-human mammal, the method comprising: providing an immunodeficient non-human mammal; injecting said mammal with a composition, said composition comprising human CD34+ progenitor cells or splenocytes isolated from another non-human mammal, wherein said another non-human mammal is a humanized non-human mammal.

In another aspect, the invention relates to a method for testing a therapeutic approach, the method comprising: providing an immunodeficient non-human mammal; injecting said mammal with a composition, said composition comprising human CD34+ progenitor cells or splenocytes isolated from another non-human mammal, wherein said another non-human mammal is a humanized non-human mammal; testing a therapy in said mammal; and evaluating the effect of said therapy in said mammal.

The invention further provides, in another aspect, for a method of testing a cancer therapy, the method comprising: providing an immunodeficient non-human mammal; injecting said mammal with a composition, said composition comprising human CD34+ progenitor cells or splenocytes isolated from another non-human mammal, wherein said another non-human mammal is a humanized non-human mammal; introducing a tumor tissue from a patient; administering a cancer therapy to said non-human mammal; and evaluating the effect of said therapy in said non-human mammal.

In yet another aspect, the invention provides a method for selecting one or more clinical trial participants from a pool of candidates, the method comprising: providing an immunodeficient non-human mammal; injecting said mammal with a composition, said composition comprising a candidate's human CD34+ progenitor cells; administering a therapy to said non-human mammal; and evaluating the immune response of the established human immune system.

The present invention also provides for a method for maintaining a human immune system in a non-human mammal, the method comprising: injecting a naïve immunodeficient mammal with splenocytes isolated from a humanized mouse; isolating splenocytes from said injected naïve immunodeficient mammal; and injecting said isolated splenocytes into a naïve immunodeficient mammal of a subsequent generation.

In another aspect, the invention provides a method for maintaining or expanding a culture of B and T leukocytes, the method comprising: introducing leukocytes from a heterogeneous mammal into a recipient mammal; isolating splenocytes of said recipient mammal after at least 4 weeks after the introduction of said leukocytes; injecting said splenocytes into a naïve immunodeficient mammal; isolating leukocytes from said injected mammal after at least 4 weeks post the injection; and isolating said heterogeneous mammal leukocytes from said leukocytes.

In another aspect, the invention provides for a method for producing B and T leukocytes, the method comprising: introducing leukocytes from a heterogeneous mammal into a recipient mammal; isolating splenocytes of said recipient mammal after at least 4 weeks after the introduction of said leukocytes; injecting said splenocytes into a naïve immunodeficient mammal; isolating leukocytes from said injected mammal after at least 4 weeks post the injection; and isolating said heterogeneous mammal leukocytes from said leukocytes. In yet another aspect, the invention provides for isolated B and T leukocytes produced by the method described herein.

In another aspect, the invention provides for a method for producing one or more animals, each comprising a population of heterologous leukocytes, the method comprising: introducing leukocytes from a heterogeneous mammal into a recipient mammal; isolating splenocytes of said recipient mammal after at least 4 weeks after the introduction of said leukocytes; and injecting said splenocytes into a naïve immunodeficient mammal.

Furthermore, in another aspect, present invention provides for a method for producing a model of immune system of a mammal having cancer, the method comprising: introducing a tumor tissue from a heterogeneous mammal into a recipient mammal; isolating splenocytes of said recipient mammal after at least 12 weeks after the introduction of said tumor tissue; and injecting said splenocytes into a naïve immunodeficient mammal.

In another aspect the present invention additionally provides for a pharmaceutical composition comprising B and T leukocytes, produced according to the methods described hereinabove.

### BRIEF DESCRIPTION OF THE DRAWINGS

FIG. 1 illustrates a flowchart of a method for humanizing mice and its therapeutic use, according to one embodiment of the invention.

FIG. 2 presents a schematic methodology for adoptive transfer of immune cells from humanized mice. For comparison, splenocytes, bone marrow and peripheral blood monocytes (PBMCs) were used.

FIG. 3 presents a graph showing flow cytometry analysis on peripheral blood of mice reconstituted with splenocytes, bone marrow or PBMCs from a humanized NOG mouse (12 weeks post reconstitution). Overall, adoptive transfer of splenocytes generated high levels of hCD45, with a robust fraction represented by human T-cells (CD3) and B-cells (CD19). Adoptive transfer of bone marrow cells generated good hCD45 reconstitution with very poor reconstitution of T-cells. Reconstitution of PBMCs was not observed

FIG. 4A presents a graph showing flow cytometry analysis on peripheral blood of mice reconstituted with splenocytes, from a humanized NOG mouse. Overall, adoptive transfer of splenocytes generated high levels of hCD45 cells. In average, 14.7%, 32% and 60.5% of viable cells were human CD45 cells at 3, 6 and 9 weeks post reconstitution, respectively.

FIG. 4B presents a graph showing flow cytometry analysis on peripheral blood of NOG mice reconstituted with splenocytes. Immune reconstitution provided robust levels of hCD45 leucocytes, with representative subsets of CD3 T-cells, CD19 B-cells and CD56 NK-cells.

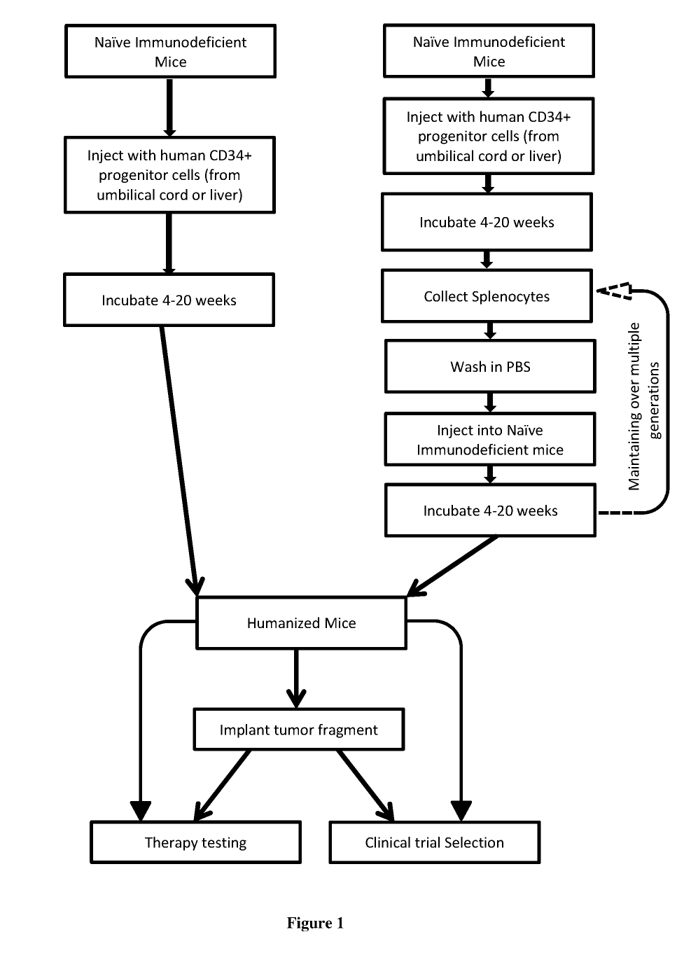

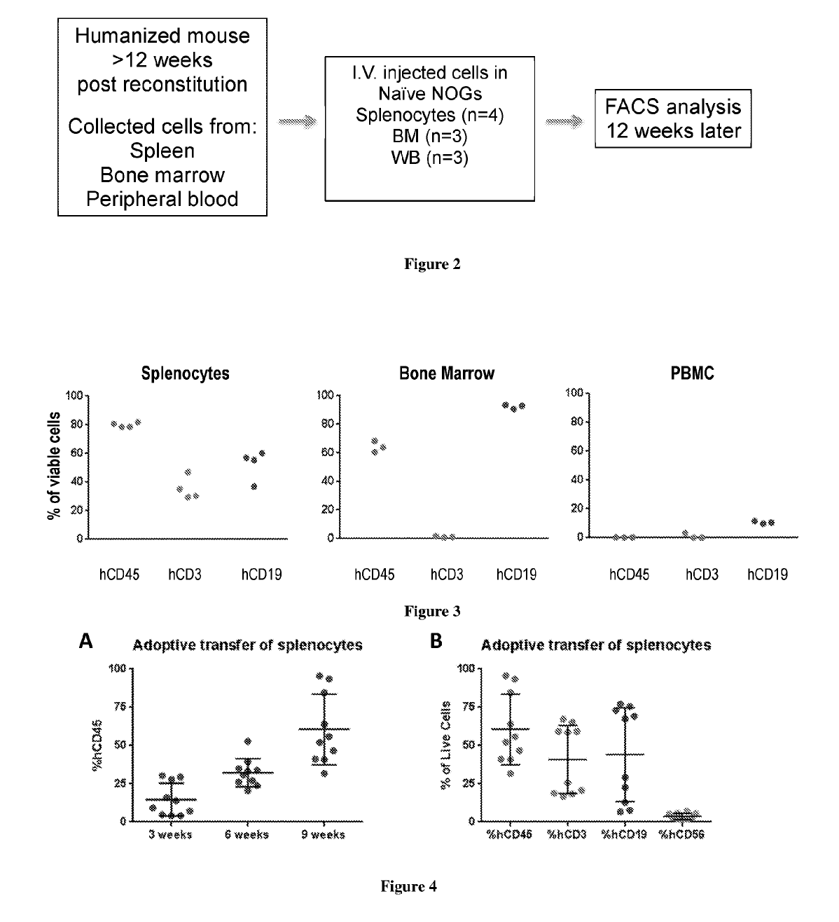

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention generally provides for a method of establishing and maintaining of a human immune system of in a non-human mammal. This invention also generally provides for a non-human mammal model comprising a human immune system. Specifically the present invention provides for a method of establishing a human subject's immune system in immunodeficient mice through administering isolated human CD34+ progenitor cells to said mice. This invention further provides for maintaining the human subject's immune system in immunodeficient mice through isolating splenocytes of mice previously administered with human CD34+ progenitor cells and administering the isolated splenocytes to one or more naïve immunodeficient mice. This invention additionally provides for the use of mice comprising a human subject immune system for testing therapeutic methods, specifically for testing cancer therapies.

In one embodiment, a method of the invention comprises the steps of isolating immune cells from a subject and administering the isolated cells into an immunodeficient non-human mammal thereby generating a “humanized” non-human mammal. The method of the present invention also comprises maintaining successive generations of humanized non-human mammals harboring a subject's immune cells.

The term “humanized”, as used herein refers to an immunodeficient mammal that harbors a population of heterogeneous immune cells that were introduced into it. The source of the heterogeneous immune cells may be either a donor mammal, or another humanized mammal.

The subject can be a human or a non-human mammal. Examples of non-human mammals include, but are not limited to, farm animals (e.g., cows, pigs, and horses), domesticated animals (e.g., dogs, cats, rabbits, and horses), human companion animals, zoo animals, wild animals, and laboratory animals (e.g., rats, mice, hamsters, guinea pigs, monkeys, and apes).

The methods of the invention further provide for isolation of the hematopoietic stem cells (HSCs) from the donor mammals. The methods of isolating the HSCs are well known in the art and include, for example, fluorescence activated cell sorting (FACS) targeting appropriate cellular markers. Suitable markers for each of these cell types are well known in the art, and, in case of human HSCs include CD34+, CD59+, Thy1/CD90+, C-kit/CD117+. In a preferred embodiment, the human HSCs are CD34+ HSCs. CD34+ HSC can be harvested from the subject's fetal liver, spleen or bone marrow. Each represents a separate embodiment of the invention.

This invention further provides for administration of the isolated HSCs to immunodeficient non-human mammals. HSCs can be administered to one or multiple immunodeficient mammals. Where HSCs are administered to several different immunodeficient mammals, these mammals may be of the same species or of different species to explore the effectiveness of establishing immune system in various species.

This invention further provides for the use of immunodeficient recipient non-human mammals. The recipient non-human mammals may include dogs, cats, rabbits, rats, mice, hamsters, or guinea pigs. In a preferred embodiment, the invention provides for the use of immunodeficient mice as the recipient mammals. The term “immunodeficient” as used herein refers to an animal's impaired or otherwise not fully functioning immune system, for example an inability to produce a normal amount of B-cells, T-cells, NK-cells, etc. The immunodeficient phenotype can be, in one embodiment, a result of a naturally occurring genetic defect, or, in another embodiment, a result of an induced genetic defect. Immunodeficiency may be produced by, for example, but not limited to, mutations, irradiation, a chemical or pharmaceutical, or a virus. Examples of immunodeficient mice include nude (nu⁻/nu⁻) mice, nude and severe combined immunodeficiency (SCID) mice, non obese diabetic (NOD) mice, NOD/SCID mice, NSG (NOD/SCID/γc^{-/-}) mice, NOG (NOD/γc^{-/-}) mice, Rag-1 (rag-1^{-/-}/γc^{-/-}) mice, or Rag-2 (rag-2^{-/-}/gc^{-/-}) BRG mice (BALB/c-Rag2^{null}/IL2rγ^{null}), Rag 1^{-/-} mice, Rag 1^{-/-}/γc^{-/-} mice, Rag 2^{-/-} mice, and Rag 2^{-/-}γc^{-/-} mice. In a preferred embodiment, the immunodeficient mice are NOD mice carrying various mutations in the interleukin-2 receptor gamma chain (IL2Rγ) gene. Examples of such mice include NOD/SCID IL2rγ^{null} and NOD/SCID IL2rγ^{Trunc} mice. In a particularly preferred embodiment, the immunodeficient mice are NOG (Pkrdc^{scid}IL2Rγ^{tm1Sug}) mice.

The present invention provides for establishing a subject's immune system in immunodeficient mice.

After leukocytes injection into an immunodeficient mouse strain, the leukocytes migrate via the recipient's vascular system into mouse tissue, most notably the spleen and bone marrow (described in Simpson-Abelson et al., 2008, The Journal of Immunology, 180, 7009, which is incorporated herein by reference in its entirety). These cells retain their ability to differentiate and are capable of expansion after tumor injection (see Bernard et al., 2008, Clinical and Experimental Immunology, 152, 406 which is incorporated herein by reference in its entirety). Thus the invention provides for harvesting of splenocytes after the heterologous subject's immune system has been established and the leukocytes migration into spleen has taken place. An immune system can be considered “established” after it has been given an appropriate amount of time to develop in the animal after inoculation of the HSCs into the animal. The time allowed for the tissue for developing in the animal is referred to as an “establishment period.” In another embodiment, the establishment period is 7-15 weeks. In another embodiment, the establishment period is 8-14 weeks. In another embodiment, the establishment period is 9-13 weeks. In another embodiment, the establishment period is 10-12 weeks. In another embodiment, the establishment period is 8-15 weeks. In another embodiment, the establishment period is 9-15 weeks. In another embodiment, the establishment period is 10-15 weeks. In another embodiment, the establishment period is 12-15 weeks. In another embodiment, the establishment period is 7-15 weeks. In another embodiment, the establishment period is 13-15 weeks. In another embodiment, the establishment period is 14-15 weeks. In another embodiment, the establishment period is 6-7 weeks. In another embodiment, the establishment period is 6-8 weeks. In another embodiment, the establishment period is 6-9 weeks. In another embodiment, the establishment period is 6-10 weeks. In another embodiment, the establishment period is 6-11 weeks. In another embodiment, the establishment period is 6-12 weeks. In another embodiment, the establishment period is 6-13 weeks. In another embodiment, the establishment period is 6-14 weeks. In another embodiment, the establishment period is 8-10 weeks. In another embodiment, the establishment period is 9-11 weeks. In another embodiment, the establishment period is 10-12 weeks. In another embodiment, the establishment period is 11-13 weeks. In another embodiment, the establishment period is 12-14 weeks. In another embodiment, the establishment period is 13-15 weeks. In another embodiment, the establishment period is 7 weeks. In another embodiment, the establishment period is 8 weeks. In another embodiment, the establishment period is 9 weeks. In another embodiment, the establishment period is 10 weeks. In another embodiment, the establishment period is 11 weeks. In another embodiment, the establishment period is 13 weeks. In another embodiment, the establishment period is 14 weeks. In another embodiment, the establishment period is 15 weeks. In another embodiment, the establishment period more than 15 weeks. In a preferred embodiment, the establishment period is 12 weeks.

In another preferred embodiment the establishment period is determined experimentally. The immune system can be considered to be “established” when the mouse humanized with human CD34+ HSCs is capable of providing mature leukocytes. For example detection of mature leukocytes in the recipient mammal's peripheral blood or organs such as spleen or bone marrow is indicative of immune system having been established and migration having taken place. The methods of detecting the target cells are well known in the art and include, but not limited to immunohistochemistry, fluorescent in situ hybridization (FISH), fluorescence activated cell sorting (FACS) targeting appropriate cellular markers. For example for human T cells the suitable markers comprise human CD45, CD3, CD4, CD8 and TCR, or a combination thereof; for human B cells suitable markers comprise anti-human CD45, CD19, IgM, or a combination thereof; for human myeloid cells suitable markers comprise human CD45, Mac-1, Gr-1, CD16, CD56, MHC Class II, or a combination thereof; for human NK cells suitable markers comprise human CD45, CD16, CD56, or a combination thereof; for human NKT cells suitable markers comprise CD45, CD3, CD4, CD8, CD16, CD56, or a combination thereof. Alternatively the maturation of leukocytes can be ascertained through detection of specific nucleic acids or proteins in routine biochemical assays, such as PCR or immunoblotting.

The methods of the invention provide for harvesting of leukocytes from a one or more of recipient's tissues. In one embodiment the leukocytes are harvested from the recipient's lungs. In another embodiment the leukocytes are harvested from the recipient's kidney. In another embodiment the leukocytes are harvested from the recipient's intestine. In a preferred embodiment the leukocytes are harvested from the recipient's peripheral blood. In a preferred embodiment the leukocytes are harvested from the recipient's bone marrow. In a particularly preferred embodiment the leukocytes are harvested from the recipient's spleen (splenocytes).

“Harvesting” refers to removing the organ containing the cells of interest from the host animal, such as the recipient mammal and disrupting the structure of said organ sufficiently to release individual cells. Methods of harvesting leukocytes from various organs are well known in the art. For example splenocytes can be collected through mechanical disruption of the spleen by forcing the excised spleen tissue through a cell strainer or nylon mesh followed by centrifugation (see e.g. .Reeves and Reeves. 2001, Removal of Lymphoid Organs. Current Protocols in Immunology. 1:III:1.9:1.9.1-1.9.3.)

In some embodiments the leukocytes can be further enriched or isolated from the pool of harvested cells using flow cytometry, such as FACS. This technique has the advantage of being able to simultaneously isolate phenotypically pure populations of viable leukocytes for molecular analysis and subsequent use. Thus different subsets leukocytes can be isolated and analyzed for activation status, anti-tumor activity, and drug resistance.

The harvested splenocytes may be also propagated in in vitro culture. The methods of culturing splenocytes are well known in the art. Furthermore, the present invention also contemplates additional manipulation of harvested splenocytes, such as stimulation with human or non-human cytokines or antigens, or genetic manipulation such as modulating activity of endogenous genes through well-known techniques, or introducing heterologous genes into splenocytes using methods that are well known in the art.

“Enriched”, as in an enriched population of cells, can be defined based upon the increased number of cells having a particular marker in a fractionated set of cells as compared with the number of cells having the marker in the unfractionated set of cells.

“Isolated” refers to a cell that is removed from its natural environment (such as in a solid tumor) and that is isolated or separated, and is at least about 75% free, and most preferably about 90% free, from other cells with which it is naturally present, but which lack the marker based on which the cells were isolated.

In the preferred embodiment of the invention the above method is used to harvest and optionally enrich splenocytes. The resulting cell population in one embodiment comprises subject's T cells. In another embodiment, the resulting cell population consists of subject's T cells. In another embodiment, the resulting population comprises subject's B cells. In another embodiment, the resulting population consists of subject's B cells. In another embodiment the resulting population comprises a mixture of subject's T cells and B cells. In another embodiment the resulting population consists of a mixture of subject's T cells and B cells. In yet another embodiment the resulting population comprises additional types of leukocytes.

In one embodiment, leukocytes comprise at least about 50% of the harvested recipient splenocytes. In another embodiment, leukocytes comprise at least about 55% of the harvested recipient splenocytes. In another embodiment, leukocytes comprise at least about 60% of the harvested recipient splenocytes. In another embodiment, leukocytes comprise at least about 65% of the harvested recipient splenocytes. In another embodiment, leukocytes comprise at least about 70% the harvested recipient splenocytes. In another embodiment, leukocytes comprise at least about 75% of the harvested recipient splenocytes. In another embodiment, leukocytes comprise at least about 80% of the harvested recipient splenocytes. In another embodiment, leukocytes comprise at least about 85% of the harvested recipient splenocytes. In another embodiment, leukocytes comprise at least about 90% of the harvested recipient splenocytes. In another embodiment, leukocytes comprise at least about 95% of the harvested recipient splenocytes. In another embodiment, leukocytes comprise at least about 96% of the harvested recipient splenocytes. In another embodiment, leukocytes comprise at least about 97% of the harvested recipient splenocytes. In another embodiment, leukocytes comprise at least about 98% of the harvested recipient splenocytes. In another embodiment, leukocytes comprise at least about 99% of the harvested recipient splenocytes. In another embodiment, leukocytes comprise 100% of the harvested recipient splenocytes.

In one embodiment, T cells comprise at least about 5% of harvested leukocytes. In another embodiment, T cells comprise at least about 10% of harvested leukocytes. In another embodiment, T cells comprise at least about 15% of harvested leukocytes. In another embodiment, T cells comprise at least about 20% of harvested leukocytes. In another embodiment, T cells comprise at least about 25% of harvested leukocytes. In another embodiment, T cells comprise at least about 30% of harvested leukocytes. In another embodiment, T cells comprise at least about 35% of harvested leukocytes. In another embodiment, T cells comprise at least about 40% of harvested leukocytes. In another embodiment, T cells comprise at least about 46% of harvested leukocytes. In another embodiment, T cells comprise at least about 50% of harvested leukocytes.

In one embodiment, B cells comprise at least about 5% of harvested leukocytes. In another embodiment, B cells comprise at least about 10% of harvested leukocytes. In another embodiment, B cells comprise at least about 15% of harvested leukocytes. In another embodiment, B cells comprise at least about 20% of harvested leukocytes. In another embodiment, B cells comprise at least about 25% of harvested leukocytes. In another embodiment, B cells comprise at least about 30% of harvested leukocytes. In another embodiment, B cells comprise at least about 36% of harvested leukocytes. In another embodiment, B cells comprise at least about 40% of harvested leukocytes. In another embodiment, B cells comprise at least about 45% of harvested leukocytes. In another embodiment, B cells comprise at least about 50% of harvested leukocytes. In another embodiment, B cells comprise at least about 55% of harvested leukocytes. In another embodiment, B cells comprise at least about 60% of harvested leukocytes. In another embodiment, B cells comprise at least about 65% of harvested leukocytes. In another embodiment, B cells comprise at least about 70% of harvested leukocytes.

In one embodiment, the harvested T cells are CD3⁺CD8⁺ T cells. In another embodiment, the harvested T cells are CD3⁺CD4⁺ T cells. In another embodiment, harvested T cells are CD45RO⁺ memory T cells. In another embodiment, harvested T cells are CD11a⁺ memory T cells. In another embodiment harvested T cells are CXCR3⁺ memory T cells. In another embodiment, harvested T cells are CD44⁺ memory T cells. In another embodiment, harvested T cells are CD69⁻ memory T cells. In another embodiment, harvested T cells are CD69L⁻ memory T cells. In another embodiment, harvested T cells are CD25⁻ memory T cells. In another embodiment, harvested T cells are CD4⁺ FOXP3⁺ regulatory T cells (T_{reg}). In another embodiment, harvested T cells are CD4⁺ FOXP3⁻ regulatory T cells (T_{reg}). In another embodiment the harvested T cells comprise a mixture of some or all types of T cells described above.

In one embodiment, the harvested B cells are CD19⁺CD20⁺ B cells. In another embodiment, harvested B cells are CD78⁺ CD138⁺ plasma cells. In another embodiment, harvested B cells are CD27⁺ memory B cells. In another embodiment, harvested B cells are CD20⁺CD27⁺CD43⁺CD70⁻ B-1 cells. In another embodiment the harvested B cells comprise a mixture of some or all types of B cells described above.

The present invention furthermore provides for cryopreservation of harvested recipient splenocytes or enriched leukocytes. The methods of splenocytes cryopreservation are well known in the art (see e.g. Gad et al., 2013, Journal for ImmunoTherapy of Cancer 1(Suppl 1), 211). The present invention contemplates numerous uses of cryopreserved tumor-associated leukocytes, including, but not limited to administration to a naïve immunodeficient mammal as described below, or in treatment of metastatic disease.

The present invention further provides for administering the splenocytes harvested from humanized mammal or enriched leukocytes to a naïve immunodeficient mammal. The naïve immunodeficient mammal can be chosen for a particular application, and can be any suitable mammal known to one of skill for the particular application. In a preferred embodiment, the recipient mammal is a mouse. In some embodiments the naïve immunodeficient mammal is the same species as the humanized mammal from which splenocytes were isolated. In some embodiments the naïve immunodeficient mammal is a different species than the humanized mammal from which splenocytes were isolated. In another embodiment, the naïve immunodeficient mammal is the same species as the subject. In another embodiment, the naïve immunodeficient mammal is a different species than the subject. In one embodiment the administering the splenocytes harvested from humanized mammal or enriched leukocytes are administered to multiple naïve immunodeficient mammals, thereby expanding of the in vivo culture of subject's leukocytes.

The invention provides for administration of a fixed number of harvested recipient mammal splenocytes or enriched leukocytes to the naïve immunodeficient mammal. In one embodiment, at least about 10⁵ cells are administered to a naïve immunodeficient mammal. In another embodiment, at least about 2×10⁵ cells are administered to a naïve immunodeficient mammal. In another embodiment, at least about 3×10⁵ cells are administered to a naïve immunodeficient mammal. In another embodiment, at least about 4×10⁵ cells are administered to a naïve immunodeficient mammal. In another embodiment, at least about 5×10⁵ cells are administered to a naïve immunodeficient mammal. In another embodiment, at least about 6×10⁵ cells are administered to a naïve immunodeficient mammal. In another embodiment, at least about 7×10⁵ cells are administered to a naïve immunodeficient mammal. In another embodiment, at least about 8×10⁵ cells are administered to a naïve immunodeficient mammal. In another embodiment, at least about 9×10⁵ cells are administered to a naïve immunodeficient mammal. In another embodiment, at least about 10⁶ cells are administered to a naïve immunodeficient mammal. In another embodiment, at least about 1.2×10⁶ cells are administered to a naïve immunodeficient mammal. In another embodiment, at least about 1.4×10⁶ cells are administered to a naïve immunodeficient mammal. In another embodiment, at least about 1.5×10⁶ cells are administered to a naïve immunodeficient mammal. In another embodiment, at least about 1.6×10⁶ cells are administered to a naïve immunodeficient mammal. In another embodiment, at least about 1.8×10⁶ cells are administered to a naïve immunodeficient mammal. In another embodiment, at least about 2×10⁶ cells are administered to a naïve immunodeficient mammal. In another embodiment, at least about 2.2×10⁶ cells are administered to a naïve immunodeficient mammal. In another embodiment, at least about 2.4×10⁶ cells are administered to a naïve immunodeficient mammal. In another embodiment, at least about 2.5×10⁶ cells are administered to a naïve immunodeficient mammal. In some embodiments, the number of administered cells is determined from the wait of the naïve immunodeficient mammal.

The invention further provides for washing of harvested splenocytes or enriched leukocytes prior to administration into naïve immunodeficient mammal. Washing solutions comprise saline, serum-free culture medium or any other solution that may be deemed suitable by a skilled artisan.

The invention further provides for expansion of the in vivo culture of leukocytes in the naïve immunodeficient mammals post-administration. In one embodiment this is achieved through administering harvested recipient mammal splenocytes or enriched leukocytes to multiple naïve immunodeficient mammals. In one embodiment, harvested recipient mammal splenocytes or enriched leukocytes are administered to 2 naive immunodeficient mammals. In another embodiment, harvested recipient mammal splenocytes or enriched leukocytes are administered to 3 naive immunodeficient mammals. In another embodiment, harvested recipient mammal splenocytes or enriched leukocytes are administered to 4 naive immunodeficient mammals. In another embodiment, harvested recipient mammal splenocytes or enriched leukocytes are administered to 5 naive immunodeficient mammals. In another embodiment, harvested recipient mammal splenocytes or enriched leukocytes are administered to 6 naive immunodeficient mammals. In another embodiment, harvested recipient mammal splenocytes or enriched leukocytes are administered to 7 naive immunodeficient mammals. In another embodiment, harvested recipient mammal splenocytes or enriched leukocytes are administered to 8 naive immunodeficient mammals. In another embodiment, harvested recipient mammal splenocytes or enriched leukocytes are administered to 9 naive immunodeficient mammals. In another embodiment, harvested recipient mammal splenocytes or enriched leukocytes are administered to 10 naive immunodeficient mammals. In another embodiment, harvested recipient mammal splenocytes or enriched leukocytes are administered to more than 10 naive immunodeficient mammals.

In another embodiment the expansion of the culture of tumor associated leukocytes in the naïve immunodeficient mammals post-administration is achieved through extending the time between administration and subsequent harvesting. In another embodiment, the expanded cultures are harvested 7-15 weeks post administration. In another embodiment, the expanded cultures are harvested 8-14 weeks post administration. In another embodiment, the expanded cultures are harvested 9-13 weeks post administration. In another embodiment, the expanded cultures are harvested 10-12 weeks post administration. In another embodiment, the expanded cultures are harvested 8-15 weeks post administration. In another embodiment, the expanded cultures are harvested 9-15 weeks post administration. In another embodiment, the expanded cultures are harvested 10-15 weeks post administration. In another embodiment, the expanded cultures are harvested 12-15 weeks post administration. In another embodiment, the expanded cultures are harvested 7-15 weeks post administration. In another embodiment, the expanded cultures are harvested 13-15 weeks post administration. In another embodiment, the expanded cultures are harvested 14-15 weeks post administration. In another embodiment, the expanded cultures are harvested 6-7 weeks post administration. In another embodiment, the expanded cultures are harvested 6-8 weeks post administration. In another embodiment, the expanded cultures are harvested 6-9 weeks post administration. In another embodiment, the expanded cultures are harvested 6-10 weeks post administration. In another embodiment, the expanded cultures are harvested 6-11 weeks post administration. In another embodiment, the expanded cultures are harvested 6-12 weeks post administration. In another embodiment, the expanded cultures are harvested 6-13 weeks post administration. In another embodiment, the expanded cultures are harvested 6-14 weeks post administration. In another embodiment, the expanded cultures are harvested 8-10 weeks post administration. In another embodiment, the expanded cultures are harvested 9-11 weeks post administration. In another embodiment, the expanded cultures are harvested 10-12 weeks post administration. In another embodiment, the expanded cultures are harvested 11-13 weeks post administration. In another embodiment, the expanded cultures are harvested 12-14 weeks post administration. In another embodiment, the expanded cultures are harvested 13-15 weeks post administration. In another embodiment, the expanded cultures are harvested 7 weeks post administration. In another embodiment, the expanded cultures are harvested 8 weeks post administration. In another embodiment, the expanded cultures are harvested 9 weeks post administration. In another embodiment, the expanded cultures are harvested 10 weeks post administration. In another embodiment, the expanded cultures are harvested 11 weeks post administration. In another embodiment, the expanded cultures are harvested 13 weeks post administration. In another embodiment, the expanded cultures are harvested 14 weeks post administration. In another embodiment, the expanded cultures are harvested 15 weeks post administration. In another embodiment, the establishment period more than 15 weeks post administration. In a preferred embodiment, the expanded cultures are harvested 12 weeks.

The present invention can be used for treating any disease or disorder. In one aspect, the humanized non-human mammal of the invention can used for screening any disease or disorder.

In one example, the invention provides for a method of testing a cancer treatment in the background of the subject's immune system. The method of cancer treatment testing generally comprises the steps of establishing the subject's immune in a non-human mammal as described above; introducing a heterologous tumor from the subject into said non-human mammal; administering a test treatment to said non-human mammal and evaluating the effect of said treatment in said non-human mammal.

The term “cancer” refers to a proliferative disorder associated with unrestrained cell growth, uncontrolled cell proliferation, and decreased cell death via apoptosis. The term “tumor” is used herein to refer to a group of cells that exhibit abnormally high levels of growth and proliferation. A tumor may be malignant, pre-malignant, or; benign; malignant tumor cells are cancerous. The term “tumor” as used herein also refers to a portion of a tumor; for example a sample of a tumor. The term “tumor” as used herein also to refer to both primary tumors and metastases. The term “tumor growth” is used herein to refer to proliferation or growth by a cell or cells that comprise a tumor that leads to a corresponding increase in the size of the tumor. As used throughout, the terms “cancer” and “tumor” may in certain embodiments be used interchangeably, having all the same meanings and qualities.

According to this invention the heterologous tumor, can be a malignant tumor. The heterologous tumor, can also be, a benign tumor. In some cases, benign tumors may represent significant clinical problems and/or may behave like malignant tumors. Examples of such benign tumors include but are not limited to pituitary neurofibromas, neuromas, adenomas, and/or meningiomas. As contemplated by this invention, the heterologous tumor is a solid tumor. In some embodiments, the tumor is a portion of a tumor. Examples of solid tumors include, but are not limited to brain tumors, myeloblastomas, breast tumors, lymphomas, non-Hodgkin's lymphomas, head and neck tumors, bladder tumors, eye tumors, thyroid tumors, salivary gland tumors, adrenal tumors, esophageal tumors, intestinal tumors, gastric tumors, colon tumors, lung tumors, liver tumors, pancreatic tumors, kidney tumors, prostate tumors, muscular tumors, osseous tumors, skin tumors, and stromal/sarcoma tumors. In some embodiments, the tumor, or portion thereof, is a primary tumor. In some embodiments, the tumor is metastases. In some embodiments of the invention, the tumor is a human tumor. As contemplated by this invention, tumor, or portion thereof, may be derived from a cancer patient undergoing anti-cancer therapy, e.g. surgery, chemotherapy, radiation therapy, antibody therapy, immunotherapy, or any combination thereof. In other embodiments, the tumor, or portion thereof, is derived from a patient who has not undergone anti-cancer therapy.

This invention provides for introducing one or more heterologous tumors, or portions thereof into a non-human mammal wherein a subject's or a patient's immune system has been previously established. The methods of introducing heterologous tumors into mammals are well known in the art. For example the tumor can be engrafted or implanted subcutaneously. Other methods of introducing heterologous tumors have been also described in the art (see e.g. Morton and Houghton, Nature Protocols, 2, 247 (Feb. 22, 2007) and US Patent application US20140109246 A1 which are, incorporated herein by reference in their entirety). The tumor or portion thereof may be implanted orthotopically, or at the same site in the recipient mammal as the origin of the tumor. Thus, for example, a kidney tumor may be implanted in the kidney of the recipient mammal. The tumor may also be implanted heterotopically, or in a location that is different from where tumor was derived, for example, and in a preferred embodiment in the flank of the recipient mammal. This invention also provides for implantation of multiple portions of the same tumor in the same mammal, for example both orthotopically and heterotopically. In another embodiment, the portions of the same tumor may be implanted into several individual mammals, all or some of which comprise a subject's or a patient's immune system established as described above. In one embodiment, the tumor, or fragment thereof, is implanted into 2 recipient mammals. In another embodiment, the tumor, or fragment thereof, is implanted into 3 recipient mammals. In another embodiment, the tumor, or fragment thereof, is implanted into 4 recipient mammals. In another embodiment, the tumor, or fragment thereof, is implanted into 5 recipient mammals. In another embodiment, the tumor, or fragment thereof, is implanted into more than 5 recipient mammals. The tumor, or portion thereof, can be removed from the subject and implanted directly into the recipient mammal. The tumor may also be cut into small pieces prior to implantation of each piece into recipient mammal or mammals. In one embodiment, the tumor is cut into 5 mm³ pieces prior to implantation. In another embodiment, the tumor is cut into 10 mm³ pieces prior to implantation. In another embodiment, the tumor is cut into 15 mm³ pieces prior to implantation. In another embodiment, the tumor is cut into 20 mm³ pieces prior to implantation. In another embodiment, the tumor is cut into 25 mm³ pieces prior to implantation. In another embodiment, the tumor is cut into 30 mm³ pieces prior to implantation. In another embodiment, the tumor is cut into 5-30 mm³ pieces prior to implantation. In another embodiment, the tumor is cut into 10-25 mm³ pieces prior to implantation. In another embodiment, the tumor is cut into 15-20 mm³ pieces prior to implantation. In another embodiment, the tumor is cut into 10-30 mm³ pieces prior to implantation. In another embodiment, the tumor is cut into 15-30 mm³ pieces prior to implantation. In another embodiment, the tumor is cut into 20-30 mm³ pieces prior to implantation. In another embodiment, the tumor is cut into 25-30 mm³ pieces prior to implantation. In another embodiment, the tumor is cut into 5-10 mm³ pieces prior to implantation. In another embodiment, the tumor is cut into 5-15 mm³ pieces prior to implantation. In another embodiment, the tumor is cut into 5-20 mm³ pieces prior to implantation. In another embodiment, the tumor is cut into 15-20 mm³ pieces prior to implantation. In another embodiment, the tumor is cut into 10-25 mm³ pieces prior to implantation. In another embodiment, the tumor is cut into 15-25 mm³ pieces prior to implantation. In another embodiment, the tumor is cut into 20-25 mm³ pieces prior to implantation.

As contemplated by this invention the tumor may be washed prior to implantation into the recipient mammal. Washing solutions comprise saline, serum-free culture medium or any other solution that may be deemed suitable by a skilled artisan. In another embodiment, the tumors are incubated in a culture medium for one or two days prior to implantation. The incubation conditions may be selected to prevent replication of the tumor cells during incubation. In a preferred embodiment, the solid tumor is not dissociated prior to implantation. Implanting a non-dissociated tumor is important, since it preserves the non-cancerous components within the tumor, including, but not limited to, B cell, T cell, NK cells, macrophage, myofibroblasts, fibroblasts, endothelial cells, blood vessels, and/or lymph vessels.

The invention also provides for monitoring of the tumor growth after implantation. The methods of tumor growth monitoring are well known in the art. Suitable methods of monitoring tumor growth comprised analysis of size of the implanted tumor and analysis of cancer stem cells (CSCs), for example by FACS, for CD44⁺, CD24⁺ cells and/or for ADLH⁺ cells.

The present invention further provides for testing treatments after tumor implant has been established. A cancer tissue can be considered “established” after it has been given an appropriate amount of time to develop in the animal after inoculation of the tissue into the animal. In some embodiments, the tissue can be considered to be “established” after it has developed into a tissue having a size ranging from about 100 mm³ to about 300 mm³. In some embodiment, the tissue can be considered to be “established” after it has developed into a tissue having a size ranging from about 50 mm³ to about 500 mm³, from about 125 mm³ to about 250 mm³, from about 75 mm³ to about 400 mm³, or any range therein.

The treatments that can be tested in the subject's genetic background comprise pharmacotherapy, chemotherapy, radiation therapy, antibody therapy, immunotherapy or any combination thereof.

The present invention further provides for a method of selecting candidates for a clinical trial, wherein a candidate's immune system is established in a non-human mammal as described above, and subsequently a prospective treatment is administered to said mammal. Once the prospective treatment has been administered the immune response to said treatment can be evaluated, allowing for prediction of undesirable immune system-based side effects in a candidate. Subsequently the candidates whose immune system established in a non-human animal displayed negative reaction to the prospective treatment can be excluded from clinical trial.

The term “about” as used herein means in quantitative terms plus or minus 5%, or in another embodiment plus or minus 10%, or in another embodiment plus or minus 15%, or in another embodiment plus or minus 20%.

It will be understood by the skilled artisan that the term “administering” encompasses bringing a subject in contact with a composition of the present invention. Compositions may be administered by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally. In a preferred embodiment, compositions may be administered by intravenous, intra-arterial, or intra-muscular injection of a liquid preparation. Suitable liquid formulations include solutions, suspensions, dispersions, emulsions, oils and the like. In one embodiment, the compositions are administered intravenously and are thus formulated in a form suitable for intravenous administration. In another embodiment, the compositions are administered intra-arterially and are thus formulated in a form suitable for intra-arterial administration. In another embodiment, the compositions are administered intra-muscularly and are thus formulated in a form suitable for intra-muscular administration. In a particularly preferred embodiment the compositions are administered via intravenous injection.

In one embodiment, leukocytes comprise at least about 50% of cells harvested post-administration and expansion in naïve immunodeficient mammals. In another embodiment, leukocytes comprise at least about 55% of cells harvested post-administration and expansion in naïve immunodeficient mammals. In another embodiment, leukocytes comprise at least about 60% of cells harvested post-administration and expansion in naïve immunodeficient mammals. In another embodiment, leukocytes comprise at least about 65% of cells harvested post-administration and expansion in naïve immunodeficient mammals. In another embodiment, leukocytes comprise at least about 70% cells harvested post-administration and expansion in naïve immunodeficient mammals. In another embodiment, leukocytes comprise at least about 75% of cells harvested post-administration and expansion in naïve immunodeficient mammals. In another embodiment, leukocytes comprise at least about 80% of cells harvested post-administration and expansion in naïve immunodeficient mammals. In another embodiment, leukocytes comprise at least about 85% of cells harvested post-administration and expansion in naïve immunodeficient mammals. In another embodiment, leukocytes comprise at least about 90% of cells harvested post-administration and expansion in naïve immunodeficient mammals. In another embodiment, leukocytes comprise at least about 95% of cells harvested post-administration and expansion in naïve immunodeficient mammals. In another embodiment, leukocytes comprise at least about 96% of cells harvested post-administration and expansion in naïve immunodeficient mammals. In another embodiment, leukocytes comprise at least about 97% of cells harvested post-administration and expansion in naïve immunodeficient mammals. In another embodiment, leukocytes comprise at least about 98% of cells harvested post-administration and expansion in naïve immunodeficient mammals. In another embodiment, leukocytes comprise at least about 99% of cells harvested post-administration and expansion in naïve immunodeficient mammals. In another embodiment, leukocytes comprise 100% of cells harvested post-administration and expansion in naïve immunodeficient mammals.

In one embodiment, T cells comprise at least about 5% of leukocytes present in cell cultures harvested post-administration and expansion in naïve immunodeficient mammals. In another embodiment, T cells comprise at least about 10% of leukocytes present in cell cultures harvested post-administration and expansion in naïve immunodeficient mammals. In another embodiment, T cells comprise at least about 15% of leukocytes present in cell cultures harvested post-administration and expansion in naïve immunodeficient mammals. In another embodiment, T cells comprise at least about 20% of leukocytes present in cell cultures harvested post-administration and expansion in naïve immunodeficient mammals. In another embodiment, T cells comprise at least about 25% of leukocytes present cell cultures in harvested post-administration and expansion in naïve immunodeficient mammals. In another embodiment, T cells comprise at least about 30% of leukocytes present in cell cultures harvested post-administration and expansion in naïve immunodeficient mammals. In another embodiment, T cells comprise at least about 35% of leukocytes present in cell cultures harvested post-administration and expansion in naïve immunodeficient mammals. In another embodiment, T cells comprise at least about 40% of leukocytes present in cell cultures harvested post-administration and expansion in naïve immunodeficient mammals. In another embodiment, T cells comprise at least about 46% of leukocytes present in cell cultures harvested post-administration and expansion in naïve immunodeficient mammals. In another embodiment, T cells comprise at least about 50% of leukocytes present in cell cultures harvested post-administration and expansion in naïve immunodeficient mammals.

In one embodiment, B cells comprise at least about 5% of leukocytes present in cell cultures harvested post-administration and expansion in naïve immunodeficient mammals. In another embodiment, B cells comprise at least about 10% of leukocytes present in cell cultures harvested post-administration and expansion in naïve immunodeficient mammals. In another embodiment, B cells comprise at least about 15% of leukocytes present in cell cultures harvested post-administration and expansion in naïve immunodeficient mammals. In another embodiment, B cells comprise at least about 20% of leukocytes present in cell cultures harvested post-administration and expansion in naïve immunodeficient mammals. In another embodiment, B cells comprise at least about 25% of leukocytes present in cell cultures harvested post-administration and expansion in naïve immunodeficient mammals. In another embodiment, B cells comprise at least about 30% of leukocytes present in cell cultures harvested post-administration and expansion in naïve immunodeficient mammals. In another embodiment, B cells comprise at least about 35% of leukocytes present in cell cultures harvested post-administration and expansion in naïve immunodeficient mammals. In another embodiment, B cells comprise at least about 40% of leukocytes present in cell cultures harvested post-administration and expansion in naïve immunodeficient mammals. In another embodiment, B cells comprise at least about 46% of leukocytes present in cell cultures harvested post-administration and expansion in naïve immunodeficient mammals. In another embodiment, B cells comprise at least about 50% of leukocytes present in cell cultures harvested post-administration and expansion in naïve immunodeficient mammals. In another embodiment, B cells comprise at least about 55% of leukocytes present in cell cultures harvested post-administration and expansion in naïve immunodeficient mammals. In another embodiment, B cells comprise at least about 60% of leukocytes present in cell cultures harvested post-administration and expansion in naïve immunodeficient mammals. In another embodiment, B cells comprise at least about 65% of leukocytes present in cell cultures harvested post-administration and expansion in naïve immunodeficient mammals. In another embodiment, B cells comprise at least about 70% of leukocytes present in cell cultures harvested post-administration and expansion in naïve immunodeficient mammals.

In one embodiment, the T cells harvested post administration and expansion are CD3⁺CD8⁺ T cells. In another embodiment, the harvested T cells are CD3⁺CD4⁺ T cells. In another embodiment, harvested T cells are CD45RO⁺ memory T cells. In another embodiment, harvested T cells are CD11a⁺ memory T cells. In another embodiment harvested T cells are CXCR3⁺ memory T cells. In another embodiment, harvested T cells are CD44⁺ memory T cells. In another embodiment, harvested T cells are CD69⁻ memory T cells. In another embodiment, harvested T cells are CD69L⁻ memory T cells. In another embodiment, harvested T cells are CD25⁻ memory T cells. In another embodiment, harvested T cells are CD4⁺ FOXP3⁺ regulatory T cells (T_{reg}). In another embodiment, harvested T cells are CD4⁺ FOXP3⁻ regulatory T cells (T_{reg}). In another embodiment the harvested T cells comprise a mixture of some or all types of T cells described above.

In one embodiment, the B cells harvested post administration and expansion are CD19⁺CD20⁺ B cells. In another embodiment, harvested B cells are CD78⁺ CD138⁺ plasma cells. In another embodiment, harvested B cells are CD27⁺ memory B cells. In another embodiment, harvested B cells are CD20⁺CD27⁺CD43⁺CD70⁻ B-1 cells. In another embodiment the harvested B cells comprise a mixture of some or all types of B cells described above.

In one embodiment, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to increasing performance free survival or overall survival of a patient. In another embodiment, “treating” refers to stabilizing the progression of a disease. In another embodiment, “treating” refers to inducing remission. In another embodiment, “treating” refers to slowing the progression of a disease. The terms “reducing”, “suppressing” and “inhibiting” refer to lessening or decreasing.

As used herein, “treatment” refers to both therapeutic treatment and prophylactic or preventative measures, wherein the object is to prevent or lessen the targeted pathologic condition or disorder as described herein. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof.

The present invention also provides for a model of immune system of a mammal having cancer comprising a naïve immunodeficient mammal administered with a culture of leukocytes as described above. These models may can be used in determining the effect of a drug or treatment on the immune system of the subject that is the source of the tumor. For example the naïve mammals administered tumor associated leukocytes can be subjected to various treatment regimens and the impact on these leukocytes can be monitored. Use of naïve immunodeficient mammals for this purpose recapitulates the immune system of a cancer patient in a cancer-free background allowing for longer test regimens. Moreover, availability of several mammals that recapitulate a patient's immune system enables testing of several treatment regimens in parallel.

The present invention also provides for a pharmaceutical composition comprising leukocytes isolated according to the methods described above. The availability of a pharmaceutical composition comprising large numbers of leukocytes has numerous applications in the cancer patients who may frequently suffer immunodeficiency due to age, anti-cancer therapies (e.g. chemotherapy or radiation therapy), immunosuppressive drug treatment or infection. In addition such composition can be used in treatment of relapsed cancer or metastatic disease that originated from the primary tumor that was the original source of leukocytes.

As used herein the term “pharmaceutical composition” encompasses a therapeutically effective amount of the active ingredient or ingredients tumor associated leukocytes with a pharmaceutically acceptable carrier or diluent.

A “therapeutically effective amount”, in reference to the treatment of tumor, refers to an amount capable of invoking one or more of the following effects: (1) inhibition, to some extent, of tumor growth, including, slowing down and complete growth arrest; (2) reduction in the number of tumor cells; (3) reduction in tumor size; (4) inhibition (i.e., reduction, slowing down or complete stopping) of tumor cell infiltration into peripheral organs; (5) inhibition (i.e., reduction, slowing down or complete stopping) of metastasis; (6) enhancement of anti-tumor immune response, which may, but does not have to, result in the regression or rejection of the tumor; and/or (7) relief, to some extent, of one or more symptoms associated with the disorder. A “therapeutically effective amount” of tumor-associated leukocytes provided herein for purposes of treatment of tumor may be determined empirically and in a routine manner.

The term “comprise” or grammatical forms thereof, refers to the inclusion of the indicated active agent, such as the tumor-associated leukocytes of this invention, as well as inclusion of other active agents, such as an antibody or functional fragment thereof, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components, which exert a therapeutic effect via a mechanism distinct from that of the indicated active ingredient. In some embodiments, the term “consisting essentially of” may refer to components, which exert a therapeutic effect and belong to a class of compounds distinct from that of the indicated active ingredient. In some embodiments, the term “consisting essentially of” may refer to components, which exert a therapeutic effect and may be distinct from that of the indicated active ingredient, by acting via a different mechanism of action, for example. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

As used herein, the singular form “a,” “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

In the following examples, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Thus these examples should in no way be construed, as limiting the broad scope of the invention.

### EXAMPLES

### Example 1: Procedure for Adoptive Transfer of Splenocytes of Humanized Mice

### Leukocyte Expansion

Human leukocytes were obtained from spleens of humanized mice and expanded in vivo. The passaged cells were found to be viable and to have conserved the effector memory phenotype of donor cells as seen through the presence of CD45RO⁺, CD11a⁺, CXCR3⁺, CD44⁺, CD69⁻, CD62L⁻, CD25⁻ markers.

### Experimental Design

Spleens were collected from immunografted mice (minimum of 6 weeks post human immune reconstitution). Splenocytes were prepared using standard protocols. Briefly, mice spleens were cut into small pieces and pressed through a 100 μm cell strainer. Splenocytes were next washed with sterile PBS twice and an aliquot was tested for cell viability and quantification. Cells were suspended in sterile PBS at a concentration of ≤2.5 million cells per 100 uL and a max of 200 uL will be intravenously administered to each mouse. Each splenocyte preparation allows for the engraftment of 5 to 10 NOG (Prkdc^{scid}Il2rg^{tm1/Sug}) mice. Aseptic technique was observed during this entire procedure. Splenocytes can alternatively be cryopreserved in DMSO stocks for later use.

### Analysis

Immunophenotyping by flow cytometry analysis on peripheral blood of mice was performed 12 weeks after splenocyte reconstitution to identify population levels of CD45, CD3, CD19 human markers.

### Results

In average, 80% of viable cells were human CD45 cells and of these, 30-46% were human CD3 (T cells) and 36-60% human CD19 (B-cells), 12 weeks post reconstitution (FIG. 3).

Further analyses shown that the fraction CD45 cells increased with the incubation time comprising on average, 14.7%, 32% and 60.5% of viable cells at 3, 6 and 9 weeks post reconstitution, respectively (FIG. 4A). After nine weeks of incubation robust levels of CD3 T-cells, and CD19 B-cells were also observed (FIG. 4 B).

### CONCLUSION

The authors conclude that transferred splenocytes from humanized mice can be expanded in new mice, are functional and are phenotypically indistinguishable from donor T cells.

We claim:

1. A method for establishing a human immune system in a non-human mammal, the method comprising: providing an immunodeficient non-human mammal; injecting said mammal with a composition, said composition comprising human CD34+ progenitor cells or splenocytes isolated from another non-human mammal, wherein said another non-human mammal is a humanized non-human mammal, and wherein said isolated splenocytes comprise human immune cells.

2. The method of claim 1, wherein said humanized non-human mammal is a mammal comprising a human immune system established through injection of a composition comprising human CD34+ progenitor cells.

3. The method of claim 1, wherein said CD34+ progenitor cells are from human umbilical cord blood.

4. The method of claim 1, wherein said CD34+ progenitor cells are from human fetal liver.

5. The method of claim 1, wherein said CD34+ progenitor cells are from a human subject's bone marrow.

6. The method of claim 1, wherein said non-human mammal is mouse, rat, pig, rabbit, or guinea pig.

7. The method of claim 1, wherein said humanized non-human mammal is mouse, rat, pig, rabbit, or guinea pig.

8. The method of claim 1, wherein said non-human mammal and said humanized non-human mammal are the same species.

9. The method of claim 1, wherein said non-human mammal and said humanized non-human mammal are different species.

10. A non-human mammal model comprising a human immune system established in accordance with the method of claim 1.

11. The model of claim 10, wherein the established human immune system in said non-human mammal comprises human leukocytes.

12. The model of claim 11, wherein said human leukocytes in said non-human mammal comprise at least about 20% human CD45+ cells.

13. The model of claim 12, wherein said human CD45+ cells in said non-human mammal comprise at least about 5% human CD3+ T cells.

14. The model of claim 12, wherein said human CD45+ cells in said non-human mammal comprise at least about 5% human CD19+ B cells.

15. The model of claim 12, wherein said human CD45+ cells in said non-human mammal comprise at least about 1% human CD56+ NK cells.

16. The method of claim 1, wherein said isolated splenocytes are sorted to select for a human marker prior to injection into a non-human mammal, wherein said human marker is CD45+, CD3+, CD19+, or CD56+.

17. The method of claim 1, wherein said isolated splenocytes are propagated in vitro prior to injection into a recipient non-human mammal.

18. The method of claim 1, wherein said isolated splenocytes are stimulated with human cytokines prior to injection into a recipient non-human mammal.

19. A method of testing a therapeutic approach, the method comprising: establishing a human immune system in a non-human mammal in accordance with the method of claim 1; testing a therapy in said mammal; and evaluating the effect of said therapy in said mammal.

20. The method of claim 19, wherein said therapy is an immunotherapy.

21. The method of claim 20 wherein said immunotherapy is an immune checkpoint blockade therapy, a therapy by monoclonal antibody, a therapy by a small molecule, a therapy targeting immunosuppressive molecules, or a therapy by an immunotherapeutic vaccine.

22. The method of claim 19, wherein said therapy is any chemotherapy or a combination of chemotherapy and any immunetherapy.

23. A method of testing a cancer therapy, the method comprising the steps of:
a. establishing a human immune system in a non-human mammal in accordance with the method of claim 1;
b. introducing a tumor tissue from a patient;
c. administering a cancer therapy to said non-human mammal; and,
d. evaluating the effect of said therapy in said non-human mammal.

24. The method of claim 22 wherein said tumor tissue is introduced by subcutaneous engraftment, orthotopically, or by hematogenous route.

25. The method of claim 22 wherein the tumor tissue is a sample of a solid tumor selected from head and neck tumor, a brain tumor, an eye tumor, a thyroid tumor, an adrenal tumor, a salivary gland tumor, an esophageal tumor, a gastric tumor, an intestinal tumor, a colon tumor, a lung tumor, a breast tumor, a liver tumor, a pancreas tumor, a kidney tumor, a bladder tumor, a prostate tumor, a muscular tumor, an osseous tumor, a skin tumor, and a stroma/sarcoma tumor.

26. A method for selecting one or more clinical trial participants from a pool of candidates, the method comprising: establishing a human immune system in a non-human mammal in accordance with the method of claim 1 using a candidate's CD34+ progenitor cells; administering a therapy to said non-human mammal; evaluating the immune response of the established human immune system; and selecting the individuals whose model immune system did not display unfavorable response to therapy for clinical trial.

27. A method of maintaining a human immune system in a non-human mammal, the method comprising: injecting a naïve immunodeficient mammal with splenocytes isolated from a humanized mouse produced in accordance with the method of claim 2; isolating splenocytes from said injected naïve immunodeficient mammal; and injecting said isolated splenocytes into a naïve immunodeficient mammal of a subsequent generation.

28. A method for maintaining or expanding a culture of B and T leukocytes, the method comprising: introducing leukocytes from a heterogeneous mammal into a recipient mammal; isolating splenocytes of said recipient mammal after at least 4 weeks after the introduction of said leukocytes; injecting said splenocytes into a naïve immunodeficient mammal; isolating leukocytes from said injected mammal after at least 4 weeks post the injection; and isolating said heterogeneous mammal leukocytes from said leukocytes.

29. A method for producing B and T leukocytes, the method comprising: introducing leukocytes from a heterogeneous mammal into a recipient mammal; isolating splenocytes of said recipient mammal after at least 4 weeks after the introduction of said leukocytes; injecting said splenocytes into a naïve immunodeficient mammal; isolating leukocytes from said injected mammal after at least 4 weeks post the injection; and isolating said heterogeneous mammal leukocytes from said leukocytes.

30. A method for producing one or more animals comprising a population of heterologous leukocytes, the method comprising: introducing leukocytes from a heterogeneous mammal into a recipient mammal; isolating splenocytes of said recipient mammal after at least 4 weeks after the introduction of said leukocytes; and injecting said splenocytes into a naïve immunodeficient mammal.

31. A method for producing a model of immune system of a mammal having cancer, the method comprising: introducing a tumor tissue from a heterogeneous mammal into a recipient mammal; isolating splenocytes of said recipient mammal after at least 12 weeks after the introduction of said tumor tissue; and injecting said splenocytes into a naïve immunodeficient mammal.
